(19) United States
(12) Patent Application Publication
(10) Pub. No.: US 2014/0263091 A1
(43) Pub. Date: Sep. 18, 2014

(54) Fluid Sanitization Assembly And Related Methods Of Use

(71) Applicant: Hayward Industries, Inc., Elizabeth, NJ (US)

(72) Inventors: James Anthony Carter, III, Warwick, RI (US); Douglas M. Sawyer, JR., Seekonk, MA (US)

(73) Assignee: HAYWARD INDUSTRIES, INC., Elizabeth, NJ (US)

(21) Appl. No.: 14/212,044

(22) Filed: Mar. 14, 2014

Related U.S. Application Data
(60) Provisional application No. 61/792,277, filed on Mar. 15, 2013.

(51) Int. Cl. E04H 4/12; E04H 4/16; C02F 1/78; C02F 1/32

(52) U.S. Cl. CPC E04H 4/1209; C02F 1/32; E04H 4/16; C02F 1/78
USPC 210/748.12; 210/198.1; 210/192; 210/748.11; 250/428; 250/435; 250/438

(57) ABSTRACT

Improved fluid sanitization assemblies are provided. More particularly, the present disclosure provides improved fluid/water sanitization assemblies utilizing UV light and/or ozone. The UV light and/or ozone can be generated via light emitting diodes (“LEDs”) (e.g., via UV LEDs). In certain embodiments, by emitting light at a wavelength of about 250 nm to about 270 nm (e.g., 253.7 nm), the fluid sanitization assemblies thereby disinfect and/or sanitize fluid/water. The present disclosure provides for a fluid sanitization assembly including a pressure vessel configured and dimensioned to house fluid; a plurality of light emitting diodes (“LEDs”) mounted with respect to the pressure vessel, each LED mounted with respect to the pressure vessel via an optic member; wherein each optic member is configured and dimensioned to focus and direct UV light emitted from its associated LED to the fluid within the pressure vessel for sanitization purposes.

### CROSS-REFERENCE TO RELATED APPLICATIONS

This application claims the benefit of U.S. Provisional Application No. 61/792,277 filed Mar. 15, 2013, all of which is herein incorporated by reference in its entirety.

### BACKGROUND

1. Technical Field

The present disclosure relates to fluid sanitization assemblies, and more particularly, to fluid (e.g., water) sanitization assemblies utilizing ultraviolet (“UV”) light and/or ozone.

2. Background Art

In general, fluid sanitization assemblies are known. For example, assemblies for sanitizing and/or disinfecting water have been developed. Fluid (e.g., water) sanitization assemblies are useful in a myriad of different environments for various uses/applications, such as commercial and/or industrial applications. For example, some fluid sanitization assemblies are described and disclosed in U.S. Pat. Nos. 3,079,498; 3,336,099; 4,842,723; 6,991,735; 7,767,168 and 8,043,500, the entire contents of each being hereby incorporated by reference in their entireties.

Some water sanitization assemblies utilize a conventional UV bulb design, the lifespan of which degrades after time (e.g., after several thousands of hours of use). Such useful lifespan degradation is difficult to detect, typically other than by measuring the hours of run time. This can create situations where the unit/assembly appears to be working because the light may be on, but the unit/assembly is no longer capable of properly sanitizing the water. These UV lamps/bulbs also typically require a high rate of energy consumption.

Thus, an interest exists for improved fluid/water sanitization assemblies, and related methods of use. These and other inefficiencies and opportunities for improvement are addressed and/or overcome by the assemblies, systems and methods of the present disclosure.

### SUMMARY

The present disclosure provides advantageous fluid (e.g., water) sanitization assemblies. More particularly, the present disclosure provides improved fluid/water sanitization assemblies utilizing UV light and/or ozone. In exemplary embodiments, the UV light and/or ozone are generated via light emitting diodes (“LEDs”) (e.g., via UV LEDs). In certain embodiments, by emitting light at a wavelength of about 250 nm to about 270 nm (e.g., about 253.7 nm), the fluid sanitization assemblies thereby disinfect and/or sanitize fluid/water.

The present disclosure provides for a fluid sanitization assembly including a pressure vessel configured and dimensioned to house fluid; a plurality of light emitting diodes (“LEDs”) mounted with respect to the pressure vessel, each LED mounted with respect to the pressure vessel via an optic member; wherein each optic member is configured and dimensioned to focus and direct UV light emitted from its associated LED to the fluid within the pressure vessel for sanitization purposes.

The present disclosure also provides for a fluid sanitization assembly including a pressure vessel configured and dimensioned to house fluid; a plurality of ultraviolet (“UV”) light emitting diodes (“LEDs”) mounted with respect to the pressure vessel, each UV LED mounted with respect to the pressure vessel via a directional optic member; wherein each directional optic member is configured and dimensioned to focus, direct and increase the intensity of the UV light emitted from its associated UV LED to the fluid within the pressure vessel for sanitization purposes.

The present disclosure also provides for a fluid sanitization assembly further including a venturi mounted with respect to the pressure vessel, the venturi configured to introduce ozone to the fluid within the pressure vessel for sanitization purposes. The present disclosure also provides for a fluid sanitization assembly further including a plurality of UV LEDs mounted with respect to the venturi, the plurality of UV LEDs mounted with respect to the venturi configured to emit UV light to create ozone that the venturi delivers to the fluid within the pressure vessel for sanitization purposes. The present disclosure also provides for a fluid sanitization assembly wherein the plurality of UV LEDs mounted with respect to the venturi emit UV light at a wavelength of about 185 nm to create ozone.

The present disclosure also provides for a fluid sanitization assembly further including an ozone generator mounted with respect to the pressure vessel, the ozone generator configured to introduce ozone to the fluid within the pressure vessel for sanitization purposes. The present disclosure also provides for a fluid sanitization assembly wherein the plurality of UV LEDs mounted with respect to the pressure vessel emit UV light at a wavelength of from about 250 nm to about 270 nm for sanitization purposes.

The present disclosure also provides for a fluid sanitization assembly wherein the pressure vessel is mounted with respect to the piping of a swimming pool system so that fluid to be sanitized passes through the pressure vessel for sanitization purposes. The present disclosure also provides for a fluid sanitization assembly wherein each UV LED is mounted with respect to a control and power source member.

The present disclosure also provides for a fluid sanitization assembly further including an insert member disposed within the pressure vessel. The present disclosure also provides for a fluid sanitization assembly wherein the insert member is a spiraled insert member, the spiraled insert member including a reflective coating. The present disclosure also provides for a fluid sanitization assembly wherein the spiraled reflective insert member is configured to: (i) reflect UV light internally within the pressure vessel, and (ii) reduce the flow rate of the fluid within the pressure vessel.

The present disclosure also provides for a fluid sanitization assembly wherein the pressure vessel includes an inner surface, at least a portion of the inner surface including a reflective coating. The present disclosure also provides for a fluid sanitization assembly wherein each directional optic member includes a layer of light directing optical material.

The present disclosure also provides for a method for sanitizing a fluid including providing a pressure vessel configured and dimensioned to house fluid; mounting a plurality of UV LEDs with respect to the pressure vessel, with each UV LED mounted with respect to the pressure vessel via a directional optic member; providing a fluid within the pressure vessel; emitting UV light from each UV LED; wherein each directional optic member is configured and dimensioned to focus, direct and increase the intensity of the UV light emitted from its associated UV LED to the fluid within the pressure vessel for sanitization purposes.

The present disclosure also provides for a method for sanitizing a fluid wherein each UV LED emits UV light at a wavelength of from about 250 nm to about 270 nm for sanitization purposes. The present disclosure also provides for a method for sanitizing a fluid further including mounting a venturi with respect to the pressure vessel, the venturi configured to introduce ozone to the fluid within the pressure vessel for sanitization purposes.

The present disclosure also provides for a method for sanitizing a fluid further including mounting an ozone generator with respect to the pressure vessel, the ozone generator configured to introduce ozone to the fluid within the pressure vessel for sanitization purposes.

The present disclosure also provides for a method for sanitizing a fluid further including the step of mounting the pressure vessel with respect to the piping of a swimming pool system so that fluid to be sanitized passes through the pressure vessel for sanitization purposes. The present disclosure also provides for a method for sanitizing a fluid further including disposing a spiraled reflective insert member within the pressure vessel, the spiraled reflective insert member configured to: (i) reflect UV light internally within the pressure vessel, and (ii) reduce the flow rate of the fluid within the pressure vessel.

The present disclosure also provides for a fluid sanitization assembly including a pressure vessel configured and dimensioned to house fluid, the pressure vessel including an inner surface, at least a portion of the inner surface including a reflective coating; a plurality of UV LEDs mounted with respect to the pressure vessel, each UV LED mounted with respect to: (i) the pressure vessel via a directional optic member, and (ii) a control and power source member; a spiraled reflective insert member disposed within the pressure vessel; and an ozone generator mounted with respect to the pressure vessel; wherein each directional optic member is configured and dimensioned to focus, direct and increase the intensity of the UV light emitted from its associated UV LED to the fluid within the pressure vessel for sanitization purposes; wherein the plurality of UV LEDs mounted with respect to the pressure vessel emit UV light at a wavelength of from about 250 nm to about 270 nm for sanitization purposes; wherein the ozone generator is configured to introduce ozone to the fluid within the pressure vessel for sanitization purposes; and wherein the spiraled reflective insert member is configured to: (i) reflect UV light, along with the reflective coating of the inner surface of the pressure vessel, internally within the pressure vessel, and (ii) reduce the flow rate of the fluid within the pressure vessel.

Any combination or permutation of embodiments is envisioned. Additional advantageous features, functions and applications of the disclosed assemblies, systems and methods of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various steps, features and combinations of steps/features described below and illustrated in the figures can be arranged and organized differently to result in embodiments which are still within the scope of the present disclosure. To assist those of ordinary skill in the art in making and using the disclosed systems, assemblies and methods, reference is made to the appended figures, wherein:

FIG. 1 is a perspective view of a fluid sanitization assembly according to exemplary embodiments of the present disclosure;

FIG. 2 is another perspective view of the fluid sanitization assembly of FIG. 1;

FIG. 3 is another perspective view of the fluid sanitization assembly of FIG. 1;

FIG. 4 is another perspective view of the fluid sanitization assembly of FIG. 1;

FIG. 5 is another perspective view of the fluid sanitization assembly of FIG. 1;

FIG. 6 is a front view of the fluid sanitization assembly of FIG. 1;

FIG. 7 is a cross-sectional view of the fluid sanitization assembly of FIG. 6;

FIG. 8 is a side view of the fluid sanitization assembly of FIG. 1;

FIG. 9 is a cross-sectional view of the fluid sanitization assembly of FIG. 8;

FIG. 10 is a top view of the fluid sanitization assembly of FIG. 1; and

FIG. 11 is a bottom view of the fluid sanitization assembly of FIG. 1.

### DETAILED DESCRIPTION

The exemplary embodiments disclosed herein are illustrative of advantageous fluid sanitization assemblies, and systems of the present disclosure and methods/techniques thereof. It should be understood, however, that the disclosed embodiments are merely exemplary of the present disclosure, which may be embodied in various forms. Therefore, details disclosed herein with reference to exemplary fluid sanitization assemblies/fabrication methods and associated processes/techniques of assembly and/or use are not to be interpreted as limiting, but merely as the basis for teaching one skilled in the art how to make and use the advantageous fluid sanitization assemblies of the present disclosure.

The present disclosure provides improved fluid (e.g., water) sanitization assemblies. In general, the present disclosure provides improved fluid/water sanitization assemblies utilizing UV light and/or ozone. In certain embodiments, the UV light and/or ozone can be generated via light emitting diodes (“LEDs”) or the like (e.g., via UV LEDs). It is noted that by emitting light at a wavelength of about 250 nm to about 270 nm (e.g., 253.7 nm), the advantageous fluid sanitization assemblies of the present disclosure thereby disinfect and/or sanitize fluid/water. In exemplary embodiments, the present disclosure provides for a fluid sanitization assembly including a pressure vessel configured and dimensioned to house fluid; a plurality of light emitting diodes (“LEDs”) mounted with respect to the pressure vessel, each LED mounted with respect to the pressure vessel via an optic member; wherein each optic member is configured and dimensioned to focus and direct UV light emitted from its associated LED to the fluid within the pressure vessel for sanitization purposes.

Current practice provides that some water sanitization assemblies utilize a conventional UV bulb design, the lifespan of which degrades after time, and that such useful lifespan degradation is difficult to detect (e.g., other than by measuring the hours of run time). This can create situations where the assembly appears to be working (e.g., because the light may be on), but the assembly is no longer capable of properly sanitizing the water. Moreover, these UV lamps/bulbs also typically require a high rate of energy consumption. In exemplary embodiments, the present disclosure provides for improved and cost-effective fluid/water sanitization assemblies utilizing UV light and/or ozone, thereby providing a significant commercial, manufacturing and/or operational advantage as a result.

Referring now to the drawings, like parts are marked throughout the specification and drawings with the same reference numerals, respectively. Drawing figures are not necessarily to scale and in certain views, parts may have been exaggerated for purposes of clarity.

With reference to the drawings, and in particular to FIGS. 1-11, there is illustrated a fluid sanitization assembly 10 depicting an embodiment of the present disclosure. In general, fluid sanitization assembly 10 is configured and dimensioned to utilize UV light and/or ozone for sanitization purposes (e.g., to purify water). Fluid sanitization assembly 10 can be fluidically coupled to a fluid system for sanitization purposes.

For example, fluid sanitization assembly 10 may be a water sanitization assembly 10 or the like for use in sanitizing/purifying/disinfecting water (e.g., swimming pools, spas, drinking water, fish tanks, etc.). However, it is noted that exemplary fluid sanitization assembly 10 is capable of use with other fluid systems or the like for sanitization purposes (e.g., for sanitizing/purifying/disinfecting fluids, materials, containers, etc.).

Fluid sanitization assembly 10 can be coupled or plumbed to a fluid system (e.g., to the plumbing/piping of a swimming pool/spa system) so that fluid to be sanitized enters and/or passes through assembly 10 for sanitization purposes. It is noted that assembly 10 can be coupled/plumbed to a fluid system using any suitable technique/materials.

In exemplary embodiments, assembly 10 is configured and dimensioned to be coupled or plumbed in series or parallel with the plumbing/piping of a swimming pool/spa system for sanitization purposes. In general and as further discussed below, assembly 10 can then utilize UV light and/or ozone to sanitize the fluid passing through assembly 10. In certain embodiments, assembly 10 is coupled/plumbed in series or in parallel with the plumbing/piping of a swimming pool/spa system, and positioned/coupled after the filter of the swimming pool/spa system, for sanitization purposes.

In exemplary embodiments, fluid sanitization assembly 10 includes a pressure vessel or container 12. Exemplary pressure vessel 12 takes the form of a substantially cylindrical pressure vessel, although the present disclosure is not limited thereto. Rather, pressure vessel 12 may take a variety of forms.

In general, pressure vessel 12 is configured and dimensioned to house and/or allow fluid (e.g., water) to pass therethrough for sanitization purposes. It is noted that pressure vessel 12 may be configured and dimensioned to house and/or allow any suitable fluid, liquid, object and/or material to pass therethrough for sanitization purposes.

Fluid sanitization assembly 10 typically includes at least one light emitting diode (“LED”) 14. In exemplary embodiments, assembly 10 includes a plurality/array of LEDs 14. In exemplary embodiments and as shown in FIGS. 1-11, assembly 10 includes twelve LEDs. It is noted that assembly 10 can include any suitable number of LEDs 14.

In general, UV light and/or ozone can be generated via the at least one LED 14 (e.g., via a UV LED 14). In certain embodiments, with each LED 14 emitting light at a wavelength of about 250 nm to about 270 nm (e.g., about 253.7 nm), the fluid sanitization assembly 10 thereby disinfects and/or sanitizes fluid/water in pressure vessel 12 (e.g., water passing through vessel 12). By emitting light at a wavelength of about 250 nm to about 270 nm (e.g., about 253.7 nm), the LEDs 14 of assembly 10 are able to disinfect the fluid (e.g. water) through creation of free hydroxyl radicals. These radicals act as an oxidizer.

Each LED 14 is typically mounted with respect to pressure vessel 12. As shown in FIG. 6, the plurality/array of LEDs 14 can be mounted with respect to pressure vessel 12.

In one embodiment, a first side 13 of the pressure vessel 12 includes six LEDs 14 mounted with respect to the pressure vessel 12, with the six LEDs 14 of the first side 13 extending from the bottom to the top of the vessel, and a second side 15 of the vessel 12 includes six LEDs 14 mounted with respect to the vessel 12, with the six LEDs 14 of the second side extending from the bottom to the top of the vessel 12 (FIG. 6). In certain embodiments, the LEDs 14 of the first side 13 of vessel 12 are positioned about 180° around vessel 12 relative to the LEDs 14 of the second side 15 of vessel 12. However, it is again noted that assembly 10/vessel 12 can include any suitable number/positioning of LEDs 14 for sanitization purposes.

Each LED 14 is typically mounted with respect to a control and power source member 16 (e.g., a printed circuit board (“PCB”) or the like). Each control and power source member 16 is configured and adapted to provide control and/or power to each LED 14. In one embodiment and as shown in FIG. 6, the six LEDs 14 of first side 13 are mounted with respect to a first control/power source member 16, and the six LEDs 14 of second side 15 are mounted with respect to a second control/power source member 16. It is noted that each member 16 and/or LED 14 of assembly can be contained/housed within any suitable protective housing or the like.

Each LED 14 of assembly 10 is typically mounted with respect to and/or integrated with vessel 12 via an optic member 18 (e.g., directional optic member 18). As such and as shown in FIGS. 1-11, assembly 10 typically includes a plurality/array of optic members 18 (e.g., one directional optic member 18 for each LED 14 of assembly 10). In certain embodiments, each optic member 18 includes a layer of light directing optics and/or optical material. Each optic member 18 typically separates its associated LED 14 from the fluid to be sanitized in vessel 12.

In exemplary embodiments, each optic member 18 is configured and dimensioned to focus and/or increase the intensity of the light emitted from its associated LED 14 within the vessel 12. Each optic member 18 can also be configured/adapted so that the light projected within the vessel 12 reflects internally, thereby increasing the efficiency of the sanitization. Moreover, a reflective material and/or coating or the like can also be added to at least a portion of the inner surface 23 of vessel 12 to increase internal reflection and/or sanitization efficiency.

In general, each optic member 18 is configured and dimensioned to advantageously direct the light transmitted from its associated LED 14 to the fluid in (e.g., the fluid passing through) vessel 12 for sanitization purposes. As discussed further below, an insert member 20 (e.g., a reflective insert member 20) or the like may also be utilized within vessel 12 to increase the refraction of the light within vessel 12.

In exemplary embodiments, the present disclosure provides for an assembly 10 having an array of LEDs 14 that are mounted with respect to and/or are integrated with pressure vessel 12 via optic members 18 (e.g., an array of optic members 18). Similarly to that discussed above in regards to FIG. 6, a first side 13 of the vessel 12 can include six LEDs 14/optic members 18 mounted with respect to the pressure vessel 12, with the six LEDs 14/optic members 18 of the first side 13 extending from the bottom to the top of the vessel, and a second side 15 of the vessel 12 can include six LEDs 14/optic members 18 mounted with respect to the vessel 12, with the six LEDs 14/optic members 18 of the second side extending from the bottom to the top of the vessel 12 (FIG. 6). In certain embodiments, the LEDs 14/optic members 18 of the first side 13 of vessel 12 are positioned about 180° around vessel 12 relative to the LEDs 14/optic members 18 of the second side 15 of vessel 12.

However, it is again noted that assembly 10/vessel 12 can include any suitable number/positioning of LEDs 14/optic members 18 for sanitization purposes.

For example, assembly 10 can include (e.g., in lieu of or in addition to those shown in FIG. 6) one or more (e.g., an array of) inner LEDs 14/optic members 18 that are configured and dimensioned to project/transmit light outwardly from within vessel 12 to sanitize the fluid (e.g., from within the substantial center of vessel 12 and toward the outside of vessel 12).

As such, assembly 10 may include: (i) an array of peripheral LEDs 14/optic members 18 that transmit/project light inward from the outside portion/periphery of the vessel 12 and toward the center of vessel 12 (FIG. 6), and/or (ii) an array of LEDs 14/optic members 18 that transmit/project light outwardly from the inside of the vessel 12 and toward the outside of vessel 12 (e.g., and towards a reflective inner surface 23 of vessel 12—FIG. 1).

Moreover, in lieu of or in addition to the above-noted embodiments, assembly 10 may include an array of LEDs 14/optic members 18 that transmit/project light from a top side 21 of the vessel towards a bottom side 22 of the vessel (FIG. 6), and/or an array of LEDs 14/optic members 18 that transmit/project light from the bottom side 22 of the vessel towards the top side 21 of the vessel 12.

In general, the present disclosure provides for an assembly 10 having an array of LEDs 14/optic members 18, with the fluid passing in front of the array. As noted, the array of LEDs 14/optic members 18 can be projecting from the outside of the vessel 12 and towards the center of the vessel, or can be projecting from the inside of the vessel 12 towards the outside of the vessel 12 (e.g., from the inside out).

As noted above, an insert member 20 (e.g., a reflective insert member 20, such as a reflective surface in the center of vessel 12) or the like may also be utilized within vessel 12 to increase the refraction of the light within vessel 12, and/or to scatter the light and cause internal refraction. In exemplary embodiments and as shown in FIGS. 1-11, a spiraled reflective center insert member 20 can be utilized. In certain embodiments, spiraled reflective center insert member 20 is utilized to reflect light internally, while also reducing the flow rate of fluid/water through vessel 12. It is noted that while insert member 20 may take the form of a spiraled reflective center insert member 20, insert member 20 may take a variety of other suitable forms.

In exemplary embodiments and as shown in FIG. 8, a venturi 30 or the like can be utilized to draw ozone into assembly 10/vessel 12 (e.g., to introduce ozone to the fluid within the pressure vessel 12 for sanitization purposes). It is noted that the ozone can be injected from the inlet, center and/or side(s) of assembly 10. In general, the suction inlet of the venturi 30 can contain or have mounted thereon UV emitting LEDs (e.g., similar to LEDs 14). In certain embodiments, the UV emitting LEDs of the suction inlet of the venturi 30 are configured and dimensioned to emit UV light (e.g., at a wavelength of about 185 nm) to create ozone (e.g., to create ozone that the venturi 30 delivers to the fluid within the pressure vessel 12 for sanitization purposes). Moreover, optical members (e.g., similar to member 18) can be used to optimize/adjust/direct the direction of the light exiting the LEDs of the suction inlet of the venturi 30. The inlet of the venturi 30 can also be advantageously sized to substantially match the requirements for ozone production of assembly 10.

Ozone has been recognized as an effective biocide or disinfectant, and has a number of attractive features. Ozone is typically inexpensive to administer, and when ozone breaks down, it produces oxygen and a free radical oxygen atom. This oxygen free radical is a powerful oxidant. See, e.g., U.S. Pat. No. 8,354,057, the entire contents of which is hereby incorporated by reference in its entirety. In exemplary embodiments and as shown in FIG. 8, it is noted that ozone could be generated by a separate ozone generator 33 coupled or mounted with respect to the assembly 10, if desired (e.g., to introduce ozone to the fluid within the pressure vessel 12 for sanitization purposes). As shown in FIG. 8, assembly 10 can include optional venturi 30 and/or optional ozone generator 33 (e.g., optional venturi 30 and/or optional ozone generator 33 coupled or mounted with respect to the pressure vessel 12 of assembly 10).

The present disclosure provides for fluid sanitization assemblies 10 that utilize UV technology and optics, the use of which allows for a smaller and/or more energy efficient design of the exemplary assemblies 10. As such, the exemplary assemblies of the present disclosure utilize less energy consumption and/or physical space to accomplish similar sanitizing power as compared to larger conventional systems/assemblies. Moreover, the use of LED technology advantageously increases the lifespan of the exemplary assemblies 10 (e.g., by substantially eliminating the light output degradation over time).

Whereas the disclosure has been described principally in connection with fluid sanitization assemblies for use in sanitizing/purifying/disinfecting fluid/water (e.g., swimming pools, spas, drinking water, fish tanks, etc.), such description has been utilized only for purposes of disclosure and is not intended as limiting the disclosure. To the contrary, it is to be recognized that the disclosed fluid sanitization assemblies are capable of use with other sanitizing/purifying/disinfecting systems or the like (e.g., for sanitizing/purifying/disinfecting fluids, materials, containers, etc.).

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications and substitutions are intended in the foregoing disclosure. Accordingly, it is appropriate that the appended claims be construed broadly and in a manner consistent with the scope of the disclosure.

What is claimed is:

1. A fluid sanitization assembly comprising:
a pressure vessel configured and dimensioned to house fluid;
a plurality of ultraviolet (“UV”) light emitting diodes (“LEDs”) mounted with respect to the pressure vessel, each UV LED mounted with respect to the pressure vessel via a directional optic member;
wherein each directional optic member is configured and dimensioned to focus, direct and increase the intensity of the UV light emitted from its associated UV LED to the fluid within the pressure vessel for sanitization purposes.

2. The fluid sanitization assembly of claim 1 further comprising a venturi mounted with respect to the pressure vessel, the venturi configured to introduce ozone to the fluid within the pressure vessel for sanitization purposes.

3. The fluid sanitization assembly of claim 2 further comprising a plurality of UV LEDs mounted with respect to the venturi, the plurality of UV LEDs mounted with respect to the venturi configured to emit UV light to create ozone that the venturi delivers to the fluid within the pressure vessel for sanitization purposes.

4. The fluid sanitization assembly of claim 3, wherein the plurality of UV LEDs mounted with respect to the venturi emit UV light at a wavelength of about 185 nm to create ozone.

5. The fluid sanitization assembly of claim 1 further comprising an ozone generator mounted with respect to the pressure vessel, the ozone generator configured to introduce ozone to the fluid within the pressure vessel for sanitization purposes.

6. The fluid sanitization assembly of claim 1, wherein the plurality of UV LEDs mounted with respect to the pressure vessel emit UV light at a wavelength of from about 250 nm to about 270 nm for sanitization purposes.

7. The fluid sanitization assembly of claim 1, wherein the pressure vessel is mounted with respect to the piping of a swimming pool system so that fluid to be sanitized passes through the pressure vessel for sanitization purposes.

8. The fluid sanitization assembly of claim 1, wherein each UV LED is mounted with respect to a control and power source member.

9. The fluid sanitization assembly of claim 1 further comprising an insert member disposed within the pressure vessel.

10. The fluid sanitization assembly of claim 9, wherein the insert member is a spiraled insert member, the spiraled insert member including a reflective coating.

11. The fluid sanitization assembly of claim 10, wherein the spiraled reflective insert member is configured to: (i) reflect UV light internally within the pressure vessel, and (ii) reduce the flow rate of the fluid within the pressure vessel.

12. The fluid sanitization assembly of claim 1, wherein the pressure vessel includes an inner surface, at least a portion of the inner surface including a reflective coating.

13. The fluid sanitization assembly of claim 1, wherein each directional optic member includes a layer of light directing optical material.

14. A method for sanitizing a fluid comprising:
providing a pressure vessel configured and dimensioned to house fluid;
mounting a plurality of UV LEDs with respect to the pressure vessel, with each UV LED mounted with respect to the pressure vessel via a directional optic member;
providing a fluid within the pressure vessel;
emitting UV light from each UV LED;
wherein each directional optic member is configured and dimensioned to focus, direct and increase the intensity of the UV light emitted from its associated UV LED to the fluid within the pressure vessel for sanitization purposes.

15. The method of claim 14, wherein each UV LED emits UV light at a wavelength of from about 250 nm to about 270 nm for sanitization purposes.

16. The method of claim 14 further comprising mounting a venturi with respect to the pressure vessel, the venturi configured to introduce ozone to the fluid within the pressure vessel for sanitization purposes.

17. The method of claim 14 further comprising mounting an ozone generator with respect to the pressure vessel, the ozone generator configured to introduce ozone to the fluid within the pressure vessel for sanitization purposes.

18. The method of claim 14 further comprising the step of mounting the pressure vessel with respect to the piping of a swimming pool system so that fluid to be sanitized passes through the pressure vessel for sanitization purposes.

19. The method of claim 14 further comprising disposing a spiraled reflective insert member within the pressure vessel, the spiraled reflective insert member configured to: (i) reflect UV light internally within the pressure vessel, and (ii) reduce the flow rate of the fluid within the pressure vessel.

20. A fluid sanitization assembly comprising:
a pressure vessel configured and dimensioned to house fluid, the pressure vessel including an inner surface, at least a portion of the inner surface including a reflective coating;
a plurality of UV LEDs mounted with respect to the pressure vessel, each UV LED mounted with respect to: (i) the pressure vessel via a directional optic member, and (ii) a control and power source member;
a spiraled reflective insert member disposed within the pressure vessel; and
an ozone generator mounted with respect to the pressure vessel;
wherein each directional optic member is configured and dimensioned to focus, direct and increase the intensity of the UV light emitted from its associated UV LED to the fluid within the pressure vessel for sanitization purposes;
wherein the plurality of UV LEDs mounted with respect to the pressure vessel emit UV light at a wavelength of from about 250 nm to about 270 nm for sanitization purposes;
wherein the ozone generator is configured to introduce ozone to the fluid within the pressure vessel for sanitization purposes; and
wherein the spiraled reflective insert member is configured to: (i) reflect UV light, along with the reflective coating of the inner surface of the pressure vessel, internally within the pressure vessel, and (ii) reduce the flow rate of the fluid within the pressure vessel.
